(19) United States
(12) Patent Application Publication
(10) Pub. No.: US 2004/0121981 A1
(43) Pub. Date: Jun. 24, 2004

(54) Method for controlling angiogenesis in animals

(75) Inventors: Yan Chang, Ashland, MA (US); Vodek Sasak, Northboro, MA (US)

Correspondence Address: ROPES & GRAY LLP, ONE INTERNATIONAL PLACE, BOSTON, MA, 02110-2624, US

(73) Assignee: GlycoGenesys, Inc., Boston, MA (US)

(21) Appl. No.: 10/665,055

(22) Filed: Sep. 16, 2003

Related U.S. Application Data
(63) Continuation-in-part of application No. 10/299,478, filed on Nov. 19, 2002.
(60) Provisional application No. 60/331,793, filed on Nov. 21, 2001.

(51) Int. Cl. A61K 31/732

(52) U.S. Cl. 514/54

(57) ABSTRACT

Disease conditions which are dependent upon or moderated by angiogenesis are controlled by the use of a therapeutic material which interacts with cell surface galectins. Particular therapeutic materials comprise a polymeric backbone having side chains terminating in a sugar dependent therefrom. Disclosed are specific therapeutic materials in which the polymeric backbone is based upon polygalacturonic acid, and the side chains terminate in arabinose or galactose.

### CROSS-REFERENCE TO RELATED APPLICATIONS

This application is a continuation-in-part of U.S. patent application Ser. No. 10/299,478, filed on Nov. 19, 2002, which claims priority of U.S. Provisional Patent Application Serial No. 60/331,793, filed on Nov. 21, 2001, the specifications of each of which are incorporated by reference herein in their entirety.

### FIELD OF THE INVENTION

This invention relates to methods and compositions for controlling angiogenesis in an animal. More particularly, the present invention relates to materials and methods for the treatment of diseases in which angiogenesis is a factor. Most specifically, the invention relates to methods and materials for controlling angiogenesis by the use of compounds which interact with galectins such as galectin-3.

### BACKGROUND OF THE INVENTION

Medical science has recognized that angiogenesis is an important factor in the initiation and/or proliferation of a large number of diverse disease conditions. Under normal physiological conditions, humans and other animals only undergo angiogenesis in very specific, restricted situations. For example, angiogenesis is normally observed in wound healing, fetal and embryonic development, and in the formation of the corpus luteum, endometrium and placenta. The process of angiogenesis has been found to be altered in a number of disease states, and in many instances, the pathological damage associated with the disease is related to uncontrolled angiogenesis.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating new blood vessels. Creation of the new microvascular system can initiate or exacerbate disease conditions.

Persistent, unregulated angiogenesis occurs in a multiplicity of disease states, including tumor metastasis and abnormal growth by endothelial cells, and supports the pathological damage seen in these conditions. The diverse pathological states created due to unregulated angiogenesis have been grouped together as angiogenic dependent or angiogenic associated diseases. Therapies directed at control of the angiogenic processes could lead to the abrogation or mitigation of these diseases.

The art has made many attempts to develop materials and therapies which are capable of controlling angiogenesis. However, many materials which appear promising in vitro have proven to be relatively ineffective when applied in vivo. Furthermore, many such materials have been found to be unstable, toxic, or otherwise difficult to employ. Consequently, there is a need for additional methods and materials capable of controlling angiogenesis in a reliable manner.

### SUMMARY OF THE INVENTION

The present invention recognizes that galectins play a significant role in moderating angiogenesis. The invention further recognizes that compounds which interact with galectins (e.g., galectin-3) can control disease conditions in which angiogenesis plays a role.

There is disclosed herein a method for controlling angiogenesis in an organism. The method comprises administering to the organism a therapeutically effective amount of a compound which binds to a galectin (e.g., galectin-3). In specific embodiments, the therapeutically effective compound comprises a substantially demethoxylated polygalacturonic acid which is interrupted with rhamnose residues. In other instances, the compound may be characterized as a polymeric backbone having side chains dependent therefrom which side chains are terminated by a galactose or arabinose unit. In specific instances, the compound comprises a modified pectin, particularly pH-modified pectin, enzymatically modified pectin and/or thermally modified pectin.

The compound may be administered orally, nasally, transdermally, topically, or by injection or by inhalation.

In particular embodiments, the therapeutic treatment of the present invention is directed to diseases which are dependent upon neovascularization.

### BRIEF DESCRIPTION OF THE DRAWINGS

FIG. 1 shows the effect of GCS-100 on HUVEC cell migration.

FIG. 2 shows that GCS-100 inhibits HUVEC cell migration.

FIG. 3 shows that GCS-100 inhibits I¹²⁵-labeled VEGF binding to HUVEC cells.

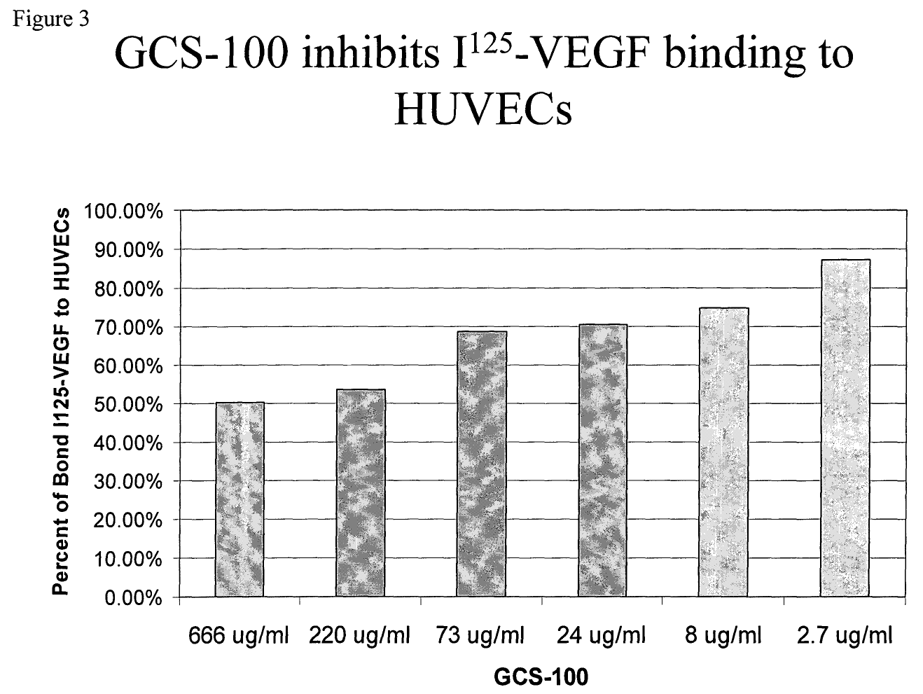

FIG. 4 shows that GCS-100 inhibits I¹²⁵-labeled VEGF binding to HUVEC cells.

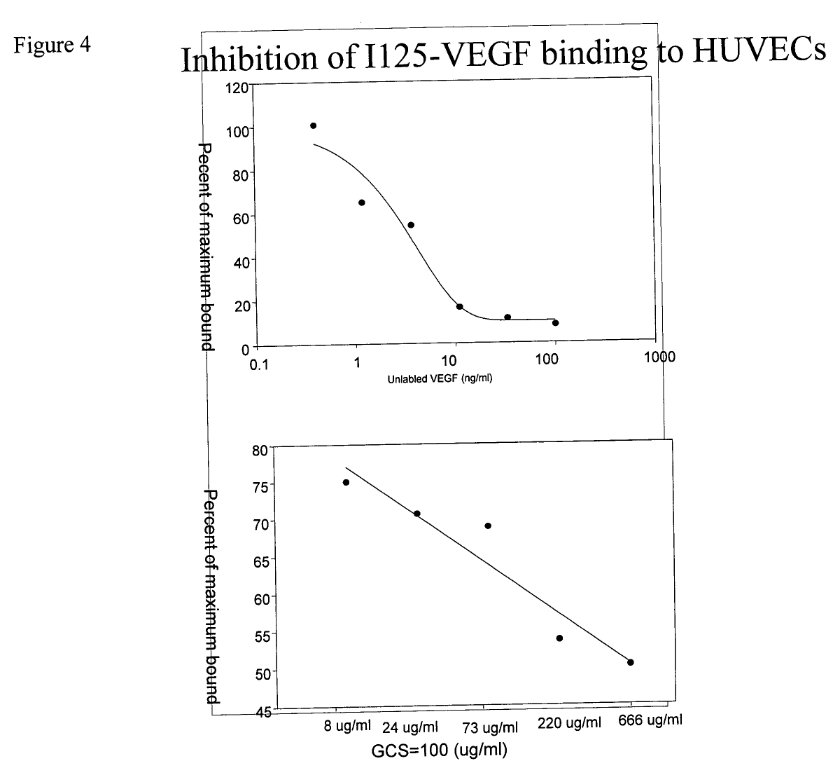

FIG. 5 shows EC migration inhibition by GCS-100.

### DETAILED DESCRIPTION OF THE INVENTION

The present invention recognizes the role of galectins in angiogenesis, and provides a therapeutic material which will advantageously interact with galectins (e.g., galectin-3) so as to moderate or prevent the manifestations of angiogenesis-dependent disease. Specifically, the present invention recognizes that particular carbohydrate materials will bind to or otherwise interact with galectins and thereby modify their interaction with cellular structures, and thereby control angiogenesis. As used herein, the term “angiogenesis” means the generation and growth of new blood vessels into a tissue or organ.

Galectins comprise a family of proteins which are expressed by plant and animal cells, and which bind β-galactoside sugars. These proteins can be found on cell surfaces, in cytoplasm, and in extracellular fluids. They have a molecular weight in the general range of 29-34 kD; they have an affinity for β-galactoside-containing materials, and have been found to play important roles in a number of biological processes. Galectin-1 and galectin-3 are specific members of this family which have been found to interact with various cellular structures, and galectin-3 has been demonstrated to promote angiogenesis in vitro.

While galectins are known to bind galactose and other such simple sugars in vitro, those simple sugars are not therapeutically effective in moderating angiogenesis in vivo. While not wishing to be bound by speculation, the inventors hereof presume that such relatively small sugar molecules are incapable of blocking, activating, suppressing, or otherwise interacting with other portions of the galectin protein (e.g., galectin-3). Therefore, preferred materials for the practice of the present invention generally comprise molecules which contain an active galectin binding sugar site, but which have somewhat higher molecular weights than simple sugars. Such molecules preferably have a minimum molecular weight of at least 300 daltons, and most typically a molecular weight in the range of 10 kD-200 kD.

A preferred class of therapeutic materials comprises oligomeric or polymeric species having one or more sugars such as galactose or arabinose pendent therefrom. The oligomeric or polymeric backbone may be synthetic or organic. Such materials will preferably have a molecular weight in the range of 3,000-150,000 daltons. It should be kept in mind that there is some inherent uncertainty in molecular weight measurements of high molecular weight carbohydrates, and measured molecular weights will be somewhat dependent on the method used for measuring the molecular weight. Molecular weights given herein are based on viscosity measurements, and such techniques are known in the art.

In certain aspects, the modified pectins of the invention are described by formulas VI and VII below, and it is to be understood that variants of these general formulae may be prepared and utilized in accord with the principles of the present invention.

Homogalacturonan

-[α-GalpA-(1→4)-α-GalpA]ₙ— (I)

Rhamnogalacturonan

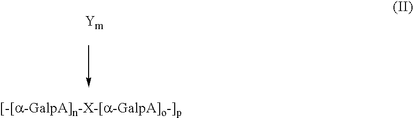

In the formulae above, m, n, o and p are ≧1, X can be either α-GalpA or α-Rhap; and Yₘ represents a side chain which may be a linear or branched chain of sugars (each Y in the chain Yₘ can independently represent a different sugar within the side chain). The sugar Y may be, but is not limited to, any of the following: α-Galp, β-Galp, β-Apif, β-Rhap, α-Rhap, α-Fucp, β-GlcpA, α-GalpA, β-GalpA, β-DhapA, Kdop, β-Acef, α-Galp, α-Arap, β-Araf, and α-Xylp.

It will be understood that natural pectin does not possess a strictly regular repeating structure, and that additional random variations are likely to be introduced by partial hydrolysis of the pectin, so that the identity of Yₘ and the values of n and o may vary from one iteration to the next of the p repeating units represented by formula II above.

Abbreviated sugar monomer names used herein are defined as follows: GalA: galacturonic acid; Rha: rhamnose; Gal: galactose; Api: erythro-apiose; Fuc: fucose; GlcA: glucuronic acid; DhaA: 3-deoxy-D-lyxo-heptulosaric acid; Kdo: 3-deoxy-D-manno-2-octulosonic acid; Ace: aceric acid (3-C-carboxy-5-deoxy-L-lyxose); Ara: arabinose. Italic p indicates the pyranose form, and italicf indicates a furanose ring.

Pectin is a complex carbohydrate having a highly branched structure comprised of a polygalacturonic backbone with numerous branching side chains dependent therefrom. The branching creates regions which are characterized as being “smooth” and “hairy.” It has been found that pectin can be modified by various chemical, enzymatic or physical treatments to break the molecule into smaller portions having a more linearized, substantially demethoxylated polygalacturonic backbone with pendent side chains of rhamnose residues having decreased branching. This material is known in the art as modified pectin, and its efficacy in treating cancer has been established. U.S. Pat. No. 5,895,784, the disclosure of which is incorporated herein by reference, describes modified pectin materials, techniques for their preparation, and use of the material as a treatment for various cancers. The material of the '784 patent is described as being prepared by a pH-based modification procedure in which the pectin is put into solution and exposed to a series of programmed changes in pH which results in the breakdown of the molecule to yield therapeutically effective modified pectin. The material in the '784 patent is most preferably prepared from citrus pectin; however, it is to be understood that modified pectins may be prepared from pectin starting material obtained from other sources, such as apple pectin and the like. Also, modification processes may be accomplished by enzymatic treatment of the pectin, or by physical processes such as heating. Further disclosure of modified pectins and techniques for their preparation and use are also disclosed in U.S. Pat. No. 5,834,442 and U.S. patent application Ser. No. 08/024,487, the disclosures of which are incorporated herein by reference. Modified pectins of this type generally have molecular weights in the range of 1-150 kD.

As disclosed in the prior art, such modified pectin materials have therapeutic efficacy against a variety of cancers. These materials interact with galectins, including galectin-1 and galectin-3, and in that regard also have efficacy in controlling diseases and conditions in which angiogenesis is a factor. In accord with the present invention, angiogenesis can be controlled or moderated by the use of modified pectin materials and other materials which interact with galectins. These materials may be administered orally; or by intravenous injection; or by injection directly into an affected tissue, as for example by injection into an arthritic joint. In some instances the materials may be administered topically, as in the form of eye drops, nasal sprays, ointments or the like. Also, other techniques such as transdermal delivery systems, inhalation or the like may be employed.

While the foregoing discussion has been primarily directed to therapeutic materials based upon modified pectins, it is to be understood that the present invention is not so limited. In accord with the general principles of the present invention, any member of the broad class of compounds which can interact with and block galectins (e.g., galectin-3) may be employed to treat angiogenesis-associated diseases. These materials, in a preferred embodiment, comprise carbohydrate materials, since such materials are low in toxicity and exhibit strong interaction with galectins. Modified pectin materials comprise one particularly preferred group of carbohydrate materials. Likewise, synthetic and semi-synthetic analogs thereof such as polygalacturonic acid materials may be similarly employed.

The compounds described above can be provided as pharmaceutically acceptable formulations using formulation methods known to those of ordinary skill in the art. These formulations can be administered by standard routes. In general, the combinations may be administered by the topical, transdermal, oral/nasal, rectal or parenteral (e.g., intravenous, subcutaneous or intramuscular) route. The combinations may be administration either by injection or by inhalation. In addition, the combinations may be incorporated into biodegradable polymers allowing for sustained release of the compound, the polymers being implanted in the vicinity of where drug delivery is desired, for example, at the site of a tumor. The biodegradable polymers and their use are described, for example, in detail in Brem et al., J. Neurosurg. 74:441-446 (1991).

The dosage of the compound will depend on the condition being treated, the particular compound, and other clinical factors such as weight and condition of the patient and the route of administration of the compound. It is to be understood that the present invention has application for both human and veterinary use. For intravenous administration to humans, a dosage of between approximately 5 to 600 mg/m²/day, preferably between approximately 80-400 mg/m²/day, and more preferably between approximately 100 to 300 mg/m²/day, is generally sufficient. For oral administration to humans, a dosage of between approximately 50 to 6000 mg/m²/day, preferably between approximately 800-4000 mg/m²/day, and more preferably between approximately 1000 to 3000 mg/m²/day, is generally sufficient.

The formulations include those suitable for oral, rectal, ophthalmic (including intravitreal or intracameral), nasal, topical (including buccal and sublingual), vaginal parenteral (including subcutaneous, intramuscular, intravenous, intradermal, intratracheal and epidural) or inhalation administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Preferred unit dosage formulations are those containing a daily dose or unit, daily sub-dose, as hereinabove recited, or an appropriate fraction thereof, of the administered ingredient.

It should be understood that in addition to the ingredients particularly mentioned above, the formulations of the present invention may include other agents conventional in the art having regard to the type of formulation in question, for example, those suitable for oral administration may include flavoring agents.

Another aspect of the invention provides aerosol formulations suitable for inhalation delivery to the respiratory tract. The respiratory tract includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli which then lead to the ultimate respiratory zone, the alveoli, or deep lung. Herein, inhalation delivery may be oral and/or nasal. Examples of pharmaceutical devices for aerosol/inhalation delivery include metered dose inhalers (MDIs), dry powder inhalers (DPIs), and air-jet nebulizers. The human lungs can remove or rapidly degrade hydrolytically cleavable deposited aerosols over periods ranging from minutes to hours. In the upper airways, ciliated epithelia contribute to the “mucociliary excalator” by which particles are swept from the airways toward the mouth. See Pavia, D., “Lung Mucociliary Clearance,” in Aerosols and the Lung: Clinical and Experimental Aspects, Clarke, S. W. and Pavia, D., Eds., Butterworths, London, 1984. In the deep lungs, alveolar macrophages are capable of phagocytosing particles soon after their deposition. See Warheit et al. Microscopy Res. Tech., 26: 412-422 (1993); and Brain, J. D., “Physiology and Pathophysiology of Pulmonary Macrophages,” in The Reticuloendothelial System, S. M. Reichard and J. Filkins, Eds., Plenum, New. York., pp. 315-327, 1985. The deep lung, or alveoli, are the primary target of inhaled therapeutic aerosols for systemic delivery.

Still another aspect of the invention relates to coated medical devices. For instance, in certain embodiments, the invention provides a medical device having a coating adhered to at least one surface, wherein the coating includes the subject compounds and preferably a polymer. Such coatings can be applied to surgical implements such as screws, plates, washers, sutures, prosthesis anchors, tacks, staples, electrical leads, valves, membranes. The devices include, but are not limited to, stents, catheters, implantable vascular access ports, blood storage bags, blood tubing, central venous catheters, arterial catheters, vascular grafts, intraaortic balloon pumps, heart valves, cardiovascular sutures, artificial hearts, a pacemaker, ventricular assist pumps, extracorporeal devices, blood filters, hemodialysis units, hemoperfasion units, plasmapheresis units, and filters adapted for deployment in a blood vessel. As discussed above, the coating according to the present invention comprises a polymer that is bioerodible or non-bioerodible. The choice of bioerodible versus non-bioerodible polymer is made based upon the intended end use of the system or device. In some embodiments, the polymer is advantageously bioerodible. For instance, where the system is a coating on a surgically implantable device, such as a screw, stent, pacemaker, etc., the polymer is advantageously bioerodible.

Although the invention contemplates using the subject carbohydrates alone, or in combination with suitable excipients, dispersing agents, and the like, in some cases, one or more compounds of the present invention are combined with monomers for forming a polymer, and are mixed to make a homogeneous solution or a homogeneous dispersion in the monomer solution. The coating is then applied to a stent or other device according to a conventional coating process. In embodiments that employ polymerizable monomers, a crosslinking process may then be initiated by a conventional initiator, such as UV light. In other embodiments that utilize polymers in conjunction with a subject carbohydrate, one or more compounds of the present invention are combined with a polymer composition to form a solution or dispersion. The dispersion is then applied to a surface of a medical device and the polymer is cross-linked to form a solid coating. In other embodiments, one or more compounds of the present invention and a polymer are combined with a suitable solvent to form a solution or dispersion, which is then applied to a stent in a conventional fashion. The solvent is then removed by a conventional process, such as heat evaporation, with the result that the polymer and the subject compounds (together forming a sustained-release drug delivery system) remain on the stent or other device as a coating.

According to the invention, a preferred device for coating is a stent. A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen. There are a multiplicity of different stents that may be utilized following coronary angioplasty. Although any number of stents may be utilized in accordance with the present invention, for simplicity, a limited number of stents will be described in exemplary embodiments. The skilled artisan will recognize that any number of stents may be utilized in connection with the present invention. In addition, as stated above, other medical devices may be utilized.

The stents of the present invention may be fabricated utilizing any number of methods. For example, the stent may be fabricated from a hollow or formed stainless steel tube that may be machined using lasers, electric discharge milling, chemical etching or other means. The stent is inserted into the body and placed at the desired site in an unexpanded form. In one exemplary embodiment, expansion may be effected in a blood vessel by a balloon catheter, where the final diameter of the stent is a function of the diameter of the balloon catheter used.

It should be appreciated that a stent in accordance with the present invention may be embodied in a shape-memory material, including, for example, an appropriate alloy of nickel and titanium or stainless steel. Structures formed from stainless steel may be made self-expanding by configuring the stainless steel in a predetermined manner, for example, by twisting it into a braided configuration. In this embodiment after the stent has been formed it may be compressed so as to occupy a space sufficiently small as to permit its insertion in a blood vessel or other tissue by insertion means, wherein the insertion means include a suitable catheter, or flexible rod.

On emerging from the catheter, the stent may be configured to expand into the desired configuration where the expansion is automatic or triggered by a change in pressure, temperature or electrical stimulation.

In certain embodiments, the compositions and methods of the present invention are useful for treating angiogenesis associated diseases and processes. Angiogenesis associated diseases include, but are not limited to, angiogenesis-dependent cancer (e.g., cancers which require neovascularization to support tumor growth), including, for example, solid tumors, blood born tumors such as leukemias, and tumor metastases; benign tumors, for example hemangiomas, acoustic neuromas, neurofibromas, trachomas, and pyogenic granulomas; inflammatory disorders such as immune and non-immune inflammation; chronic articular rheumatism and psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber 9195736_{—3} Syndrome; myocardial angiogenesis; plaque neovascularization; telangiectasia; hemophiliac joints; angiofibroma; and wound granulation and wound healing; telangiectasia psoriasis scleroderma, pyogenic granuloma, cororany collaterals, ischemic limb angiogenesis, corneal diseases, rubeosis, arthritis, diabetic neovascularization, fractures, vasculogenesis, and hematopoiesis; and disorders associated with inappropriate or inopportune invasion of vessels such as restenosis, capillary proliferation in atherosclerotic plaques and osteoporosis.

One example of a disease associated with angiogenesis is ocular neovascular disease. This disease is characterized by invasion of new blood vessels into the structures of the eye such as the retina or cornea. It is the most common cause of blindness and is involved in approximately twenty eye diseases. In age-related macular degeneration, the associated visual problems are caused by an ingrowth of chorioidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases and conditions associated with corneal neovascularization include, but are not limited to, epidemic keratoconjunctivitis, vitamin A deficiency, contact lens overwear, atopic keratitis, superior limbic keratitis, pterygium keratitis sicca, Sjogren's, acne rosacea, phylectenulosis, syphilis, Mycobacteria infections, lipid degeneration, chemical burns, bacterial ulcers, fungal ulcers, herpes simplex infections, herpes zoster infections, protozoan infections, Kaposi's sarcoma, Mooren's ulcer, Terrien's marginal degeneration, marginal keratolysis, rheumatoid arthritis, systemic lupus, polyarteritis, trauma, Wegener's sarcoidosis, scleritis, Stevens-Johnson disease, pemphigoid, radial keratotomy, and corneal graft rejection.

Diseases associated with retinal/choroidal neovascularization include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme disease, systemic lupus erythematosus, retinopathy of prematurity, Eales disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovascularization of the ankle) and diseases caused by the abnormal proliferation of fibrovascular or fibrous tissue including all forms of proliferative vitreoretinopathy.

Another disease in which angiogenesis is believed to be involved is rheumatoid arthritis. The blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Factors associated with angiogenesis may also have a role in osteoarthritis. The activation of the chondrocytes by angiogeneic-related factors contributes to the destruction of the joint. At a later stage, the angiogeneic factors would promote new bone formation. Therapeutic intervention that prevents the bone destruction could halt the progress of the disease and provide relief for persons suffering with arthritis.

Chronic inflammation may also involve pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity.

One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Therapy-resistant hemangiomas exist that cannot be treated with therapeutics currently in use.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula.

Angiogenesis is prominent in solid tumor formation and metastasis. Angiogeneic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors, and benign tumors such as acoustic neuroma, neurofibroma, trachoma and pyogenic granulomas. Prevention of angiogenesis could halt the growth of these tumors and the resultant damage to the animal due to the presence of the tumor.

It should be noted that angiogenesis has been associated with blood-borne tumors such as leukemias, any of various acute or chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in the bone marrow that give rise to leukemia-like tumors.

Angiogenesis is important in two stages of tumor metastasis. The first stage where angiogenesis stimulation is important is in the vascularization of the tumor, which allows tumor cells to enter the blood stream and to circulate throughout the body. After the tumor cells have left the primary site, and have settled into the secondary, metastasis site, angiogenesis must occur before the new tumor can grow and expand. Therefore, prevention or control of angiogenesis could lead to the prevention of metastasis of tumors and possibly contain the neoplastic growth at the primary site.

Knowledge of the role of angiogenesis in the maintenance and metastasis of tumors has led to a prognostic indicator for breast cancer. The amount of neovascularization found in the primary tumor was determined by counting the microvessel density in the area of the most intense neovascularization in invasive breast carcinoma. A high level of microvessel density was found to correlate with tumor recurrence. Control of angiogenesis by therapeutic means can lead to cessation of the recurrence of the tumors.

Angiogenesis is also involved in normal physiological processes such as reproduction and wound healing. Angiogenesis is an important step in ovulation and also in implantation of the blastula after fertilization. Prevention of angiogenesis could be used to induce amenorrhea, to block ovulation or to prevent implantation by the blastula, thereby preventing conception. In wound healing, excessive repair or fibroplasia can be a detrimental side effect of surgical procedures and may be caused or exacerbated by angiogenesis. Adhesions are a frequent complication of surgery and lead to problems such as small bowel obstruction.

Diseases associated with corneal neovascularization that can be treated according to the present invention include but are not limited to, diabetic retinopathy, retinopathy of prematurity, corneal graft rejection, neovascular glaucoma and retrolental fibroplasias, epidemic keratoconjunctivitis, vitamin A deficiency, contact lens overwear, atopic keratitis, superior limbic keratitis, pterygium keratitis sicca, Sjogren's, acne rosacea, phylectenulosis, syphilis, Mycobacteria infections, lipid degeneration, chemical burns, bacterial ulcers, fungal ulcers, herpes simplex infections, herpes zoster infections, protozoan infections, Kaposi's sarcoma, Mooren's ulcer, Terrien's marginal degeneration, marginal keratolysis, trauma, rheumatoid arthritis, systemic lupus, polyarteritis, Wegener's sarcoidosis, scleritis, Stevens-Johnson disease, pemphigoid, radial keratotomy, and corneal graft rejection.

Diseases associated with retinal/choroidal neovascularization that can be treated according to the present invention include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme disease, systemic lupus erythematosus, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovascularization of the ankle) and diseases caused by the abnormal proliferation of fibrovascular or fibrous tissue including all forms of proliferative vitreoretinopathy, whether or not associated with diabetes.

Diseases associated with chronic inflammation can be treated by the compositions and methods of the present invention. Diseases with symptoms of chronic inflammation include inflammatory bowel diseases such as Crohn's disease and ulcerative colitis, psoriasis, sarcoidosis and rheumatoid arthritis. Angiogenesis is a key element that these chronic inflammatory diseases have in common. The chronic inflammation depends on continuous formation of capillary sprouts to maintain an influx of inflammatory cells. The influx and presence of the inflammatory cells produce granulomas and thus, maintain the chronic inflammatory state. Inhibition of angiogenesis by the compositions and methods of the present invention would prevent the formation of the granulomas and alleviate the disease.

The compositions and methods of the present invention can be used to treat patients with inflammatory bowel diseases such as Crohn's disease and ulcerative colitis. Both Crohn's disease and ulcerative colitis are characterized by chronic inflammation and angiogenesis at various sites in the gastrointestinal tract. Crohn's disease is characterized by chronic granulomatous inflammation throughout the gastrointestinal tract consisting of new capillary sprouts surrounded by a cylinder of inflammatory cells. Prevention of angiogenesis by the compositions and methods of the present invention inhibits the formation of the sprouts and prevents the formation of granulomas.

Crohn's disease occurs as a chronic transmural inflammatory disease that most commonly affects the distal ileum and colon but may also occur in any part of the gastrointestinal tract from the mouth to the anus and perianal area. Patients with Crohn's disease generally have chronic diarrhea associated with abdominal pain, fever, anorexia, weight loss and abdominal swelling. Ulcerative colitis is also a chronic, nonspecific, inflammatory and ulcerative disease arising in the colonic mucosa and is characterized by the presence of bloody diarrhea.

The inflammatory bowel diseases also show extraintestinal manifestations such as skin lesions. Such lesions are characterized by inflammation and angiogenesis and can occur at many sites other than the gastrointestinal tract. The compositions and methods of the present invention are also capable of treating these lesions by preventing the angiogenesis, thus reducing the influx of inflammatory cells and the lesion formation.

Sarcoidosis is another chronic inflammatory disease that is characterized as a multisystem granulomatous disorder. The granulomas of this disease may form anywhere in the body and thus the symptoms depend on the site of the granulomas and whether the disease is active. The granulomas are created by the angiogenic capillary sprouts providing a constant supply of inflammatory cells.

The compositions and methods of the present invention can also treat the chronic inflammatory conditions associated with psoriasis. Psoriasis, a skin disease, is another chronic and recurrent disease that is characterized by papules and plaques of various sizes. Prevention of the formation of the new blood vessels necessary to maintain the characteristic lesions leads to relief from the symptoms.

Another disease which can be treated according to the present invention is rheumatoid arthritis. Rheumatoid arthritis is a chronic inflammatory disease characterized by nonspecific inflammation of the peripheral joints. It is believed that the blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis. Other diseases that can be treated according to the present invention are hemangiomas, Osler-Weber-Rendu disease, or hereditary hemorrhagic telangiectasia, solid or blood borne tumors and acquired immune deficiency syndrome.

Restenosis is another disease that can be inhibited or treated by the compositions and methods of the present invention. Restenosis is a process of smooth muscle cell (SMC) migration and proliferation at the site of percutaneous transluminal coronary angioplasty which hampers the success of angioplasty. The migration and proliferation of SMCs during restenosis can be considered a process of angiogenesis which may be controlled by the present methods. Therefore, the invention contemplates inhibition of restenosis by inhibiting angiogenesis according to the present methods in a patient following angioplasty procedures.

Similar to restenosis, atherosclerosis is a disease that is associated with inappropriate or inopportune invasion of vessels. For example, in atherosclerotic plaques, proliferation of capillaries is common and is considered a process of angiogenesis. Therefore, the compositions and methods of the present invention can be used to inhibit growth of atherosclerotic plaques.

In certain embodiments, the pharmaceutical composition of the present invention may be used alone or conjointly administered with another type of therapeutic agent for treating an inflammatory disease or condition. As used herein, the phrase “conjoint administration” refers to any form of administration in combination of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. Thus, an individual who receives such treatment can have a combined (conjoint) effect of different therapeutic compounds. Known therapeutics for treating an inflammatory disease or condition are described in medical textbooks such as Harrisons, Principles of Internal Medicine (McGraw Hill, Inc., New York). The particular therapeutic used depends on the nature of the disease or condition being treated.

Therapeutics useful in the treatment of inflammatory diseases or conditions involving infectious agents may include various antipathogen agents, i.e., antibiotics, antivirals, antifungals and antiparasitics. The type and concentration of therapeutic depends inter alia on the infectious agent causing the inflammatory disease or condition. In general, therapeutics from the group comprising antibiotics include, for example, tetracycline antibiotics; aminoglysodes; macrolides; penicillanic acid (6-APA)- and cephalosporanic acid (7-ACA)-derivatives having 6β- or 7β-acylamino groups, respectively, which are present in fermentatively, semi-synthetically or totally synthetically obtainable 6β-acylaminopenicillanic acid or 7β-acylaminocephalosporanic acid derivatives and/or 7β-acylaminocephalosporanic acid derivatives that are modified in the 3-position; and other β-lactam antibiotics of the clavam, penem and carbapenen type.

Anti-virals include zidovudine (AZT-Retrovir), zalcitabine (Hivid-ddC), dicanosine (Videx-ddI), Protease inhibitors of retroviruses, integrase inhibitors of retroviruses and others well known to those skilled in the art.

Other therapeutics useful in the treatment of inflammatory diseases or conditions include, but are not limited to, anti-inflammatory agents, or antiphlogistics. Antiphlogistics include, for example, glucocorticoids, such as, cortisone, hydrocortisone, prednisone, prednisolone, fluorcortolone, triamcinolone, methylprednisolone, prednylidene, paramethasone, dexamethasone, betamethasone, beclomethasone, fluprednylidene, desoxymethasone, fluocinolone, flumethasone, diflucortolone, clocortolone, clobetasol and fluocortin butyl ester; immunosuppressive agents; penicillamine; hydroxychloroquine; and nonsteroidal inflammation-inhibitors (NSAID) which encompass anti-inflammatory, analgesic, and antipyretic drugs such as salicyclic acid, difunisal and from substituted phenylacetic acid salts or 2phenylpropionic acid salts, such as alclofenac, ibufenac, ibuprofen, clindanac, fenclorac, ketoprofen, fenoprofen, indoprofen, fenclofenac, diclofenac, flurbiprofen, pirprofen, naproxen, benoxaprofen, carprofen and cicloprofen; oxicam derivatives, such as piroxicam; anthranilic acid derivatives, such as mefenamic acid, flufenamic acid, tolfenamic acid and meclofenamic acid, anilino-substituted nicotinic acid derivatives, such as the fenamates miflumic acid, clonixin and flunixin; heteroarylacetic acids wherein heteroaryl is a 2-indol-3-yl or pyrrol-2-yl group, such as indomethacin, oxmetacin, intrazol, acemetazin, cinmetacin, zomepirac, tolmetin, colpirac and tiaprofenic acid; idenylacetic acid of the sulindac type; analgesically active heteroaryloxyacetic acids, such as benzadac; phenylbutazone; etodolac; and nabumetone.

Other therapeutics useful in the treatment of inflammatory diseases or conditions include antioxidants. Antioxidants may be natural or synthetic. Antioxidants are, for example, superoxide dismutase (SOD), 21-aminosteroids/aminochromans, vitamin C or E, etc. Many other antioxidants are well known to those of skill in the art.

Inhibition of tumor tissue angiogenesis is a particular embodiment of the present invention because of the important role neovascularization plays in tumor growth. In the absence of neovascularization of tumor tissue, the tumor tissue does not obtain the required nutrients, slows in growth, ceases additional growth, regresses and ultimately becomes necrotic resulting in killing of the tumor. Therefore, the present invention provides compositions and method for inhibiting tumor neovascularization by inhibiting tumor angiogenesis. The present invention can also particularly effective against the formation of metastases because: (1) their formation requires vascularization of a primary tumor so that the metastatic cancer cells can exit the primary tumor; and (2) their establishment in a secondary site requires neovascularization to support growth of the metastases.

In a related embodiment, the invention contemplates the practice of the method in conjunction with other therapies such as conventional chemotherapy directed against solid tumors and for control of establishment of metastases. The administration of the subject angiogenesis inhibitor is typically conducted during or after chemotherapy, although it is preferably to inhibit angiogenesis after a regimen of chemotherapy at times where the tumor tissue will be responding to the toxic assault by inducing angiogenesis to recover by the provision of a blood supply and nutrients to the tumor tissue. In addition, it is preferred to administer the angiogenesis inhibitors after surgery, e.g. where a solid tumor has been removed, as a prophylaxis against metastases.

A wide array of conventional compounds have been shown to have anti-tumor activities. These compounds have been used as pharmaceutical agents in chemotherapy to shrink solid tumors, prevent metastases and further growth, or decrease the number of malignant cells in leukemic or bone marrow malignancies. Although chemotherapy has been effective in treating various types of malignancies, many anti-tumor compounds induce undesirable side effects. In many cases, when two or more different treatments are combined, the treatments may work synergistically and allow reduction of dosage of each of the treatments, thereby reducing the detrimental side effects exerted by each compound at higher dosages. In other instances, malignancies that are refractory to a treatment may respond to a combination therapy of two or more different treatments.

Therefore, pharmaceutical compositions of the present invention may be conjointly administered with a conventional anti-tumor compound. Conventional anti-tumor compounds include, merely to illustrate: aminoglutethimide, amsacrine, anastrozole, asparaginase, bcg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

The conventional anti-tumor compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxanes (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prednisolone); growth factor signal transduction kinase inhibitors; mitochondrial dysfunction inducers and caspase activators; and chromatin disruptors.

In certain aspects, the methods and compositions of the present invention are also useful for modulating physiological processes associated with angiogenesis, for example, ovulation, menstruation, and placentation. The angiogenesis inhibiting proteins of the present invention are useful in the treatment of disease of excessive or abnormal stimulation of endothelial cells. These diseases include, but are not limited to, intestinal adhesions, atherosclerosis, scleroderma, and hypertrophic scars, i.e., keloids. They are also useful in the treatment of diseases that have angiogenesis as a pathologic consequence such as cat scratch disease (Rochele minalia quintosa) and ulcers (Helicobacter pylori).

As described herein, any of a variety of tissues, or organs comprised of organized tissues, can support angiogenesis in disease conditions including skin, muscle, gut, connective tissue, joints, bones and the like tissue in which blood vessels can invade upon angiogenic stimuli.

Thus, in one related embodiment, a tissue to be treated is an inflamed tissue and the angiogenesis to be inhibited is inflamed tissue angiogenesis where there is neovascularization of inflamed tissue. In this class, the method contemplates inhibition of angiogenesis in arthritic tissues, such as in a patient with chronic articular rheumatism, in immune or non-immune inflamed tissues, in psoriatic tissue and the like.

In another related embodiment, a tissue to be treated is a retinal tissue of a patient with a retinal disease such as diabetic retinopathy, macular degeneration or neovascular glaucoma and the angiogenesis to be inhibited is retinal tissue angiogenesis where there is neovascularization of retinal tissue.

In an additional related embodiment, a tissue to be treated is a tumor tissue of a patient with a solid tumor, a metastases, a skin cancer, a breast cancer, a hemangioma or angiofibroma and the like cancer, and the angiogenesis to be inhibited is tumor tissue angiogenesis where there is neovascularization of a tumor tissue. Typical solid tumor tissues treatable by the present methods include lung, pancreas, breast, colon, laryngeal, ovarian, and the like tissues.

### EXAMPLES

The principles of the present invention are illustrated in an experimental series which assesses the effect of a therapeutic carbohydrate material of the present invention, in inhibiting the process of angiogenesis.

### Example 1

### GCS-100 Inhibits HUVEC Cell Migration

Chemotaxis is an integral part of angiogenesis, and this experimental series demonstrates the effect of a modified pectin material of the present invention in inhibiting angiogenesis. In a first portion of the experimental series, the effect of the chemoattractant vascular endothelial growth factor (VEGF) on human umbilical vein endothelial cells (HUVEC) was quantified. The experiment was carried out in a transwell plate, and in preparation therefor, HUVEC cells were grown to approximately 80% confluency. The cells were suspended in basal media and placed in a transwell plate on fibronectin coated membrane inserts at 50,000 cells per insert. Varying concentrations of VEGF were added to the bottom chamber of the transwell plate, and the plates incubated for 4 hours at 37° C. with a 5% CO₂ atmosphere. Following incubation, the membranes were fixed and stained. Nonmigrated cells were removed by mechanical abrasion and cells that migrated through the membrane were counted. Data from this first experiment is shown in Table 1. As will be seen, VEGF is a chemotactic agent which induces cell migration, which process is crucial to angiogenesis. Based upon the first experimental series, it was found that VEGF concentrations of 10-30 ng/ml produce a strong chemotactic effect. Three runs were made. Data from the experiment is summarized in Table 1 below.

TABLE 1

| | | 1 ng/ml | 3 ng/ml | 10 ng/ml | 30 ng/ml | 100 ng/ml |
|---|---|---|---|---|---|---|
| Samples | NEG. | VEGF | VEGF | VEGF | VEGF | VEGF |
| Cell count | 123 | 607 | 950 | 1144 | 898 | 1650 |
| Cell count | 300 | 766 | 1136 | 938 | 1448 | 901 |
| Cell count | 250 | 830 | | 1573 | 1140 | 1078 |
| AVER- | 224 | 734 | 1043 | 1218 | 1162 | 1210 |
| AGE | | | | | | |

In a second portion of the experiment, the effect of a therapeutic carbohydrate material of the present invention, in moderating chemotaxis, and hence angiogenesis, was evaluated. The material comprised a modified pectin which is commercially available from GlycoGenesys, Inc. of Boston, Mass., under the designation GCS-100. In this experimental series, HUVEC cells were incubated in a transwell plate with VEGF, and varying concentrations of the therapeutic material, under conditions as described above. The concentration of VEGF was 30 ng/ml. In one group of experiments, cells were incubated with VEGF in the absence of the carbohydrate material, and these experiments served as a positive control. In another group of experiments, cells were incubated with growth medium and no VEGF or therapeutic carbohydrate, and this group served as a negative control. In the remaining experiments, concentrations of the GCS-100 ranging from 0.001% to 0.1% were employed. The data from this experimental series is summarized in Table 2 below.

TABLE 2

| | VEGF 30 ng/ml | Medium | VEGF 30 ng/ml | VEGF 30 ng/ml | VEGF 30 ng/ml | VEGF 30 ng/ml | VEGF 30 ng/ml |
|---|---|---|---|---|---|---|---|
| Samples | Only | Only | GCS-100 0.001% | GCS-100 0.005% | GCS-100 0.01% | GCS-100 0.05% | GCS-100 0.1% |
| Cell count | 1322 | 208 | 841 | 750 | 463 | 364 | 271 |
| Cell count | 1167 | 346 | 819 | 539 | 412 | 594 | 222 |
| Cell count | | | 548 | 655 | | 430 | 170 |
| AVERAGE | 1244 | 277 | 736 | 648 | 437 | 463 | 221 |

The GCS-100 strongly inhibited cell migration, and the inhibition is concentration dependent. As established by this experimental series, GCS-100 is a potent inhibitor of the angiogenic process, and as such will have utility in the treatment of diseases in which angiogenesis is a factor. GCS-100 is known to bind to galectins which are found on the surface of cells such as HUVEC cells; therefore, other such carbohydrate materials which bind to galectins will be expected to exert a similar effect in inhibiting cell migration and angiogenesis.

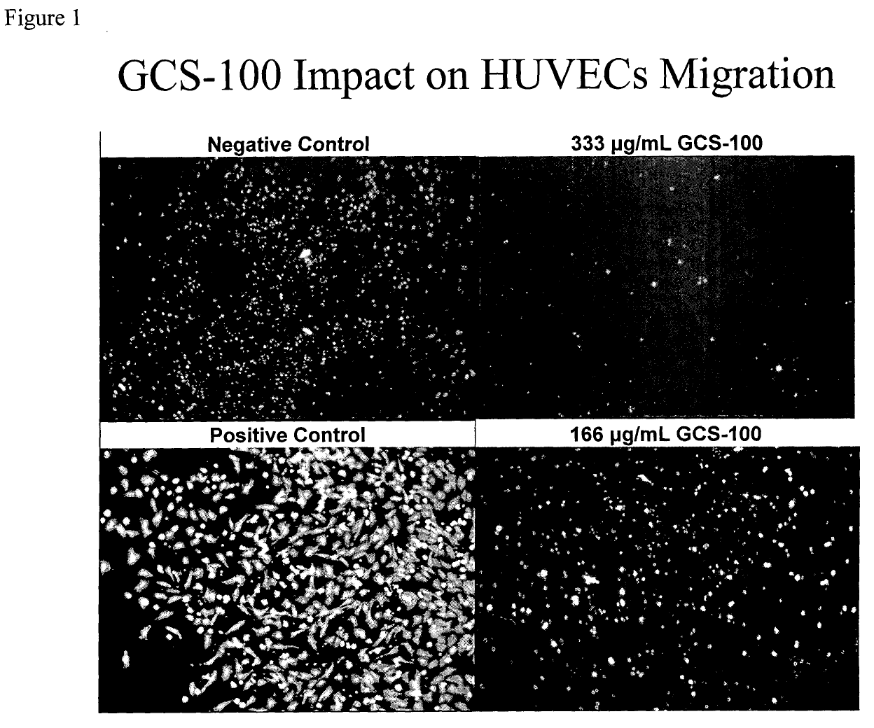

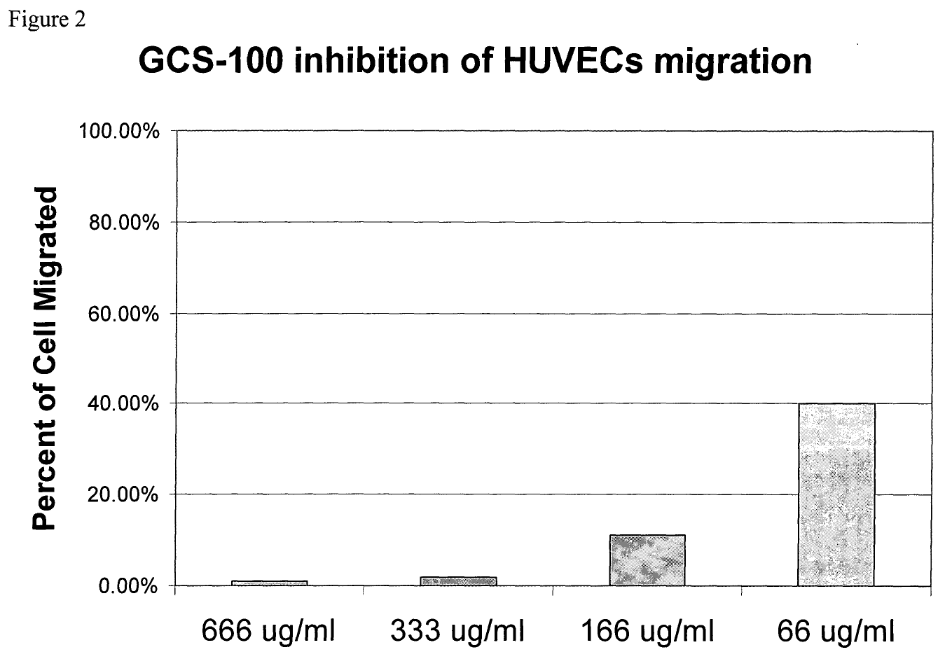

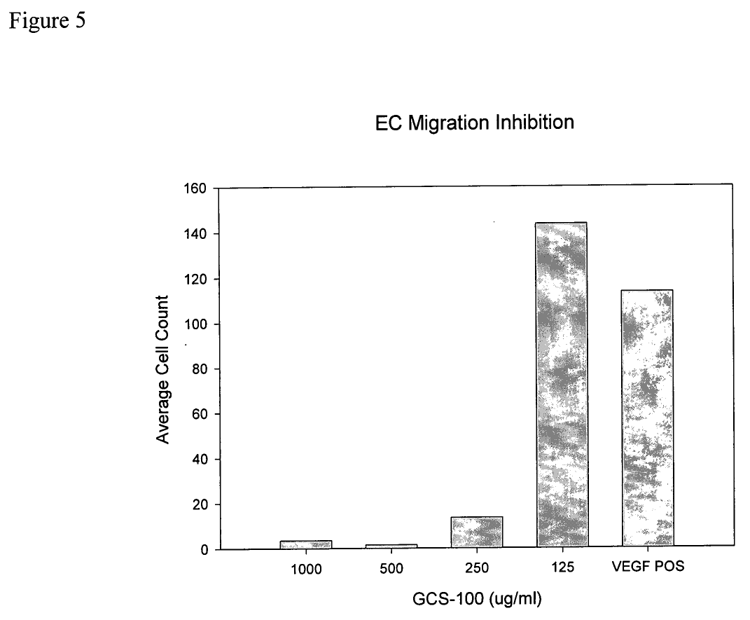

In addition, GCS-100 was shown to inhibit HUVEC cell migration in a dose-dependent manner (see FIGS. 1, 2, 5). For example, GCS-100 effectively inhibited endothelial cell migration at concentrations between 1000 and 125 ug/ml (106 and 10⁵ ng/ml).

### EXAMPLE 2

### GCS-100 Regulates Binding of ¹²⁵I-VEGF to HUVEC on 24 Well Plates

Approximately 10⁴ cells/well were incubated with inhibitors (cold VEGF or GCS-100, Lot 121340) for 1 hr at room temperature, then ¹²⁵I VEGF (1 ng/well) was added. Mixtures were incubated overnight at 4° C. Fluids were aspirated and washed 2× with 0.5 ml buffer. Triton X-100 (0.4 ml of 2% in water) was added and incubated for 30 min at room temperature and 300 ul from each well was measured in a gamma-counter). The data is shown in Table 3 below.

TABLE 3

| | | CPM | | | Avg | Minus NS | % of Max bound |
|---|---|---|---|---|---|---|---|
| | VEGF (ng/ml) | | | | | | |
| Plate 1 | 100 | 330 | 352 | 426 | 369 | 157 | 8.1 |
| | 33.3 | 440 | 426 | 418 | 428 | 216 | 11.1 |
| | 11.1 | 636 | 634 | 312 | 527 | 315 | 16.3 |
| | 3.7 | 976 | 1044 | 1766 | 1262 | 1050 | 54.1 |
| | 1.2 | 1830 | 1496 | 1086 | 1471 | 1259 | 64.9 |
| | 0.4 | 2284 | 1964 | 2242 | 2163 | 1951 | 100.6 |
| | GCS (ng/ml) | | | | | | |
| Plate 2 | 1 × 10⁶ | 2830 | 3386 | 3162 | 3126 | 2914 | 50.2 |
| | 3.3 × 10⁵ | 3292 | 3508 | 3166 | 3322 | 3110 | 53.6 |
| | 1.1 × 10⁵ | 4358 | 4048 | 4212 | 4206 | 3994 | 68.8 |
| | 3.7 × 10⁴ | 4618 | 4128 | 4182 | 4309 | 4097 | 70.6 |
| | 1.2 × 10⁴ | 4786 | 3944 | 4980 | 4570 | 4358 | 75.0 |
| | 4.1 × 10³ | 5366 | 5206 | 5250 | 5274 | 5062 | 87.2 |
| Plate 1 | | | | | | | |
| VEGF Non Specific Binding | 500 | 196 | 232 | 208 | 212 | 0 | |
| | 0 | 2476 | 2110 | 1870 | 2152 | 1940 | |
| Plate 2 | | | | | | | |
| Preincubation | ¹²⁵I-VEGF + GCS | 4088 | 4724 | 5142 | 4651 | 4439 | 76.4 |
| Maximum plate 2 | ¹²⁵I-VEGF | 5690 | 6360 | 6008 | 6019 | 5807 | 100.0 |
| | No Cells | 220 | 164 | 174 | | | |
| Total | in 400 | 60004 | 64250 | | | | |
| Total in 300 | | 46595 | | | | | |

Preincubation of GCS-100 with ¹²⁵I-VEGF decreased the amount of bound ¹²⁵I VEGF in a dose-dependent manner (see Table 3). For example, preincubation of GCS-100 (666 ug/ml) with ¹²⁵I-VEGF decreased the amount of bound VEGF by 50% compared with a control.

In summary, GCS-100 exhibited an apparent Ki that was 3×10⁵ times that of unlabeled VEGF. Fifty percent of maximum ¹²⁵I-VEGF binding was inhibited by 1×10⁶ ng/ml GCS-100 (approximately 10 μmole/L assuming average molecular weight of 90,000). Unlabeled VEGF inhibited 50% of maximal binding at 3 ng/ml (70 pmole/L).

Note that for the ¹²⁵I-VEGF binding assay, labeled VEGF was used at 2.5 ng/ml. However, in the migration experiments, VEGF was at a concentration of 20 ng/ml in the lower chamber and the cells responded to a concentration gradient.

The foregoing is illustrative of particular embodiments and features of the present invention. In view of the teaching presented herein, one of skill in the art could readily prepare and select other materials for use in controlling angiogenesis and disease conditions. The foregoing drawings, disclosure, examples and discussion are not limiting upon the present invention but are illustrative of the principles thereof. It is the following claims, including all equivalents, which define the scope of the invention.

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

1. A method for controlling angiogenesis in an organism, said method comprising:
administering to said organism a therapeutically effective amount of a compound which binds to a galectin.

2. The method of claim 1, wherein said galectin is present on the cell surface of a tissue of said organism.

3. The method of claim 1, wherein said compound binds to galectin-1 or galectin-3.

4. The method of claim 1, wherein said compound comprises a substantially demethoxylated polygalacturonic acid which is interrupted with rhamnose residues.

5. The method of claim 1, wherein said compound comprises a polymeric backbone having side chains dependent therefrom, said side chains being terminated by a galactose or arabinose unit.

6. The method of claim 1, wherein said compound comprises a modified pectin.

7. The method of claim 6, wherein said modified pectin comprises a pH modified pectin.

8. The method of claim 6, wherein said modified pectin comprises an enzymatically modified pectin.

9. The method of claim 6, wherein said modified pectin comprises a thermally modified pectin.

10. The method of claim 6, wherein said modified pectin comprises a modified citrus pectin.

11. The method of claim 6, wherein said modified pectin has a molecular weight in the range of 1-150 kilodalton.

12. The method of claim 1, wherein administering said compound to said organism comprises injecting said compound into said organism.

13. The method of claim 1, wherein administering said compound to said organism comprises topically applying said compound to said organism.

14. The method of claim 1, wherein administering said compound to said organism comprises administering said compound transdermally.

15. The method of claim 1, wherein administering said compound to said organism comprises orally administering said compound.

16. The method of claim 1, wherein administering said compound to said organism comprises administering said compound by inhalation.

17. A method for the therapeutic treatment of a disease in an animal, the progress of which disease is dependent upon neovascularization in the tissues of said animal, said method comprising:
administering to said animal a therapeutically effective amount of a compound which binds to a galectin; whereby said compound decreases the rate of angiogenesis and neovascularization in said tissues.

18. The method of claim 17, wherein said compound binds to galectin-1 or galectin-3.

19. The method of claim 17, wherein said compound comprises a substantially demethoxylated polygalacturonic acid which is interrupted with rhamnose residues.

20. The method of claim 17, wherein said compound comprises a polymeric backbone having side chains dependent therefrom, said side chains being terminated by a galactose or arabinose unit.

21. The method of claim 17, wherein said compound comprises a modified pectin.
